(19) United States
(12) Patent Application Publication
(10) Pub. No.: US 2008/0108549 A1
(43) Pub. Date: May 8, 2008

(54) Actin proteins as biomarkers for indication and targeting of resistance and sensitivity to an Abl kinase inhibitor in patients with chronic myelogenous leukemia

(76) Inventors: Ira Leonard Goldknopf, The Woodlands, TX (US); Essam Ahmed Sheta, The Woodlands, TX (US); Hagop M. Kantarjian, Bellaire, TX (US)

Correspondence Address: Power3 Medical Products, Inc., 3400 Research Forest Drive, The Woodlands, TX, 77381, US

(21) Appl. No.: 11/731,019

(22) Filed: Mar. 29, 2007

Related U.S. Application Data
(60) Provisional application No. 60/787,792, filed on Mar. 31, 2006.

(51) Int. Cl. A61K 38/02; C07K 14/435; G01N 33/53; G01N 27/26; A61P 35/00; G01N 33/50

(52) U.S. Cl. 514/2; 530/350; 436/86; 436/501; 204/456

(57) ABSTRACT

The invention relates to 5 identified protein biomarkers, gamma- and beta-Actin proteins, for screening, diagnosis, drug targeting, and drug design for resistance of cancer to an Ab1 kinase inhibitor. The method is based on the use of two-dimensional (2D) gel electrophoresis to separate the complex mixture of proteins found in bone marrow aspirate samples, taken from patients at time of diagnosis of Chronic Myelogenous Leukemia (CML), the quantitation of 5 protein spots identified as beta- and/or gamma-Actin proteins, to differentiate between patients who will respond to or resist treatment when the patients are subsequently treated with an Ab1 kinase inhibitor.

### CROSS REFERENCE TO RELATED APPLICATIONS

This application claims priority to U. S. Provisional patent application Ser. No. 60/787,792 filed Mar. 31, 2006 and entitled “Biomarkers for Diagnosis and Targeting of Resistance and Sensitivity to Imatinib Mesylate in Chronic Myelogenous Leukemia” by inventors Ira L. Goldknopf, et al.

### BACKGROUND OF THE INVENTION

It is unclear why some patients develop resistance to Ab1 kinase inhibitors such as imatinib mesylate or other anti-cancer agents, and what can be done to prevent or delay the onset of resistance. With regard to imatinib, resistance has been associated with several mechanisms including 1) amplification or mutations of the BCR-ABL fusion gene (Shah, N P, et al. 2002, Cancer Cell 2: 117-125; Gorre, M E, et al. 2001, Science 293: 876-880; Branford S, et al. 2002, Blood 99: 3472-3475; Hochhaus A, et al. 2002, Leukemia 6: 2190-2196), 2) inactivation by binding to α-1 acid glycoprotein (Gambacorti-Passerini C, et al. 2000, J. Natl. Cancer Inst. 92: 1641-1650; Gambacorti-Passerini C, et al. 2002, Blood 100: 367-368; Le Coutre Pet al. 2002 Blood Cells Mal. Dis. 28 : 75-85), and 3) increased usage of signal transduction pathways that are BCR-ABL independent. However these pathways remain undefined.

Previously, the ability to predict which patients are, or will become, resistant to a particular therapy has been limited. The ability to predict a patient's response to therapy would be a valuable asset in developing treatment strategies. For example, a patient who is identified as being resistant to imatinib could be treated with an alternative therapy or with more intensive imatinib therapy (e.g., higher dosage and/or in combination with other therapeutic agents).

1. Field of Invention

This invention relates to five protein biomarkers, gamma- and beta-Actin proteins, in bone marrow aspirates, to detect resistance to treatment of patients with cancer to an Ab1 kinase inhibitor. The method is based on the use of two-dimensional (2D) gel electrophoresis to separate the complex mixture of proteins found in bone marrow aspirate samples, taken from patients at time of diagnosis of chronic myelogenous leukemia (CML), the quantitation of 5 protein spots identified as beta- and/or gamma-Actin proteins, to differentiate between patients who will respond to or resist treatment when the patients are subsequently treated with an Ab1 kinase inhibitor.

2. Description of the Related Art

There has been a tremendous interest in the potential ability of proteomic technology to fulfill the unmet needs of effective strategies for early diagnosis (Alaiya, A. et al. 2005, J. Proteome Res. 4: 1213-1222), as well as predicting and overcoming resistance to chemotherapy of cancer. In particular, proteomics has been applied with a special emphasis on biological fluids from patients, including patients with ovarian cancer (Emmanuel F. Petricoin, A. M. Ardekani, B. A. Hitt et al. 2002, Lancet 359: 572-577) and breast cancer (Paweletz C. P. et al 2001, Dis. Markers 17: 301-307; Henry M. Kuerer, H. M. et al. 2002, Cancer 95: 2276-2282). Proteomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of proteins already known to be involved in certain biological functions has progressed from research applications alone to use in disease screening and diagnosis for clinicians. However, proteomic testing for diagnostic purposes remains in its infancy.

Detection of abnormalities in the genome of an individual can reveal the risk or potential risk for individuals to develop a disease. The transition from gene based risk to emergence of disease can be characterized as an expression of genomic abnormalities in the proteome. In fact, whether arising from genetic, environmental, or other factors, the appearance of abnormalities in the proteome signals the beginning of the process of cascading effects that can result in the deterioration of the health of the patient. Therefore, detection of proteomic abnormalities at an early stage is desired in order to allow for detection of disease processes either before the disease is established or in its earliest stages where treatment may be more effective.

Recent progress using a novel form of mass spectrometry called surface enhanced laser desorption and ionization time of flight (SELDI-TOF) for the testing of ovarian cancer and Alzheimer's disease has led to an increased interest in proteomics as a diagnostic tool (Petrocoin, E. F. et al. 2002. Lancet 359:572-577, Lewczuk, P. et al. 2004. Biol. Psychiatry 55:524-530). Furthermore, proteomics has been applied to the study of breast cancer through use of 2D gel electrophoresis and image analysis to study the development and progression of breast carcinoma in patients' breast ductal fluid specimens ((Kuerer, H. M. et al. 2002. Cancer 95:2276-2282) and in plasma (Goufman, et al. 2006, Biochemistry 2006, 71(4):354-60). In the case of breast cancer, breast ductal fluid specimens were used to identify distinct protein expression patterns in bilateral matched pair ductal fluid samples of women with unilateral invasive breast carcinoma (Kuerer, H. M. et al. 2002).

Detection of biomarkers is an active field of research. For example, U.S. Pat. No. 5,958,785 discloses a biomarker for detecting long-term or chronic alcohol consumption. The biomarker disclosed is a single biomarker and is identified as an alcohol-specific ethanol glycoconjugate. U.S. Pat. No. 6,124,108 discloses a biomarker for mustard chemical injury. The biomarker is a specific protein band detected through gel electrophoresis and the patent describes use of the biomarker to raise protective antibodies or in a kit to identify the presence or absence of the biomarker in individuals who may have been exposed to mustard poisoning. U.S. Pat. No. 6,326,209 B1 discloses measurement of total urinary 17 ketosteroid-sulfates as biomarkers of biological age. U.S. Pat. No. 6,693,177 B1 discloses a process for preparation of a single biomarker specific for O-acetylated sialic acid and useful for diagnosis and outcome monitoring in patients with lymphoblastic leukemia.

Two-dimensional (2D) gel electrophoresis has been used in research laboratories for biomarker discovery since the 1970's (Margolis J. et al. 1969, Nature. 1969 221: 1056-1057; Orrick, L. R. et al. 1973; Proc Nat'l Acad. Sci. USA. 70: 1316-1320; Goldknopf, I. L. et al. 1975, J Biol Chem. 250: 7182-7187; Goldknopf, I. L. et al. 1977, Proc Nat'l Acad Sci USA. 74: 5492-5495; O'Farrell, P. H. 1975, J. Biol. Chem. 250: 4007-4021; Anderson, L. 1977, Proc Nat'l Aced Sci USA. 74: 864-868; Klose, J. 1975, Human Genetic. 26: 231-243). The advent of much faster identification of proteins spots by in-gel digestion and mass spectroscopy ushered in the accelerated development of proteomic science through large-scale application of these techniques (Aebersold R. 2003, Nature, 422: 198-207; Kuruma, H. et al. 2004, Prostate Cancer and Prostatic Disease 1: 1-8; Kuncewicz, T. et al. 2003, Molecular & Cellular Proteomics 2: 156-163). With the advent of bioinformatics, progression of proteomics towards diagnostics and personalized medicine has become feasible (White, C. N. et al. 2004 Clinical Biochemistry, 37: 636-641; Anderson N. L. et al. 2002, Molecular & Cellular Proteomics 1:845-867). Clinical proteomics is maturing fast into a powerful approach for comprehensive analyses of disease mechanisms and disease markers (Kuruma, H. et al. 2004; Sheta, E. A. et al. 2006, Expert Rev. Proteomics 3: 45-62). We have recently applied 2D gel proteomics of human serum combined with discriminant biostatistics to the differential diagnosis of neurodegenerative diseases (Goldknopf, I. L. et al. 2006, Biochem. Biophys. Res. Commun. 342: 1034-1039; Sheta, E. A. et al. 2006). In the present invention, we use the same approach to monitor the concentrations of 5 protein biomarkers, resolved and quantitated by 2D gel electrophoresis of bone marrow aspirate samples from patients diagnosed with chronic myelogenous leukemia, to distinguish between patients who have the potential to respond from patients who have the potential to resist subsequent treatment with an Ab1 kinase inhibitor. For the purpose of illustration of the invention, the Ab1 kinase inhibitor employed is Imatinib mesylate

Although reliable individual diagnostic, prognostic, and predictive tools are limited at present, proteomics may provide new indicators and drug targets for malignancies. For example, 2D gel electrophoresis of proteins from lymphoblasts of patients with Acute Lymphocytic Leukemia (ALL) was used to identify polypeptides that could distinguish between the major subgroups of ALL (Hanash S M, et al. 1986, Proc. Natl. Acad. Sci. USA 83: 807-811). Voss et al demonstrated that B-CLL patient populations with shorter survival times exhibited changed levels of redox enzymes, HSP27, and protein disulfide isomerase, as determined by 2D gel electrophoresis of proteins prepared from mononuclear cells (Voss T, et al. 2001, Int. J. Cancer 91: 180-186). While such studies indicate that proteomics is a useful tool for the study of cancer, there remains a need for improved biomarkers and tests for identifying patients who are resistant or are likely to become resistant to a particular cancer therapy. Additionally, there is a need for improved biomarkers and targets for the treatment of drug resistant cancers.

### SUMMARY OF THE INVENTION

The present invention relates to 5 protein biomarkers in bone marrow, for determining which cancer patients have the potential for susceptibility or resistance to an Ab1 kinase inhibitor. More specifically, the present invention consists of up to 5 protein biomarkers, electrophoretic variants of gamma- and beta-Actin proteins, and their use in diagnostic assays for differentiating between chronic myelogenous leukemia patients who have the potential for susceptibility from those who have the potential for resistance to treatment with an Ab1 kinase inhibitor, for example with imatinib mesylate; for the monitoring of their therapy for early detection of development of resistance; and for new drug targets and designs to more effectively treat the resistant patients with an Ab1 kinase inhibitor, for example imatinib mesylate. The method comprises collecting a biological sample from patients having bone marrow aspirate biopsy confirmed chronic myelogenous leukemia, wherein the bone marrow aspirate samples were taken at time of initial diagnosis of chronic myelogenous leukemia, the quantitation of 5 protein spots identified as beta- and/or gamma-Actin in the bone marrow aspirate samples, the comparison of patients undergoing subsequent imatinib mesylate treatment by determining whether the patients responded or failed to respond to imatinib mesylate, to differentiate between patients who will respond to or resist treatment when the patients are subsequently treated with imatinib mesylate, based upon the concentration of the 5 protein biomarkers in the patients' pre-treatment bone marrow aspirate samples.

One aspect of the present invention is the use of up to 5 biomarkers for pre-screening a patient for potential susceptibility or resistance to an Ab1 kinase inhibitor. The method comprises collecting a biological sample from patients having bone marrow aspirate biopsy confirmed chronic myelogenous leukemia, wherein the bone marrow aspirate samples were taken at time of initial diagnosis of chronic myelogenous leukemia, the quantitation of up to 5 protein spots identified as beta- and/or gamma-actin in the bone marrow aspirate samples, and determining whether or not the patient has the potential to respond to or resist treatment with an Ab1 kinase inhibitor, based on the concentration of up to 5 protein spots identified as beta- and/or gamma-actin in the bone marrow aspirate samples. This aspect of the invention can be used as an early screen to select patients for treatment who will respond to an Ab1 kinase inhibitor, for example imatinib mesylate, and treat the potentially resistant patients with a different drug that may be more effective for them than an Ab1 kinase inhibitor. Such a screen may also be used to decide to treat some potentially resistant patients with bone marrow transplants rather than with an Ab1 kinase inhibitor. Such a screen may also be used to select patients with the potential for resistance to an Ab1 kinase inhibitor so that they may receive an additional drug to overcome resistance when and if it develops.

Another aspect of the present invention is the use of up to 5 biomarkers to determine early during treatment with an Ab1 kinase inhibitor, whether a patient is responding or developing resistance to an Ab1 kinase inhibitor. The method comprises collecting a biological sample from patients having bone marrow aspirate biopsy confirmed chronic myelogenous leukemia, wherein the bone marrow aspirate samples were taken during treatment of chronic myelogenous leukemia with an Ab1 kinase inhibitor, the quantitation of up to 5 protein spots identified as beta- and/or gamma-actin in the bone marrow aspirate samples, and determining whether the patient is developing the potential for resistance to treatment with an Ab1 kinase inhibitor, based on the concentration of up to 5 protein spots identified as beta- and/or gamma-actin in the bone marrow aspirate samples. This aspect of the invention can be used during treatment with an Ab1 kinase inhibitor as an early indication of increased risk that a patient will develop resistance to an Ab1 kinase inhibitor during further treatment. This aspect can be used to decide to initiate treatment with a different drug that may be more effective for them than an Ab1 kinase inhibitor or in combination with an Ab1 kinase inhibitor to increase the effectiveness of an Ab1 kinase inhibitor. Such a test may also be used to decide early to treat some resistant patients with bone marrow transplants before they reach blast crisis due to the development of resistance.

Another aspect of the present invention is the use of up to 5 biomarkers for determining the biological mechanism of resistance of a patient to an Ab1 kinase inhibitor and/or the drug target and/or drug design for treatment of Ab1 kinase resistant cancer. The method includes: collecting a biological sample from a patient, determining the concentrations of up to 5 protein biomarkers identified as beta- and/or gamma-actin in the biological sample, and determining the mechanism of resistance active in the patient and/or identifying the drug target appropriate for treatment, and/or designing a drug for the target for treatment of the resistant patient's cancer, based on the concentration in up to 5 protein biomarkers identified as beta- and/or gamma-actin in the bone marrow aspirate samples and the known function of gamma- and/or beta-Actin with respect to the drug target of an Ab1 kinase inhibitor.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiments disclosed might be readily utilized as a basis for modifying or redesigning the methods for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1: Representative 2D gel electrophoretic images of bone marrow aspirate samples taken prior to treatment from a patient who subsequently responded to the Ab1 kinase inhibitor imatinib mesylate (A. Sensitive) and a patient who subsequently did not respond to imatinib mesylate treatment (B. Resistant). The 5 protein biomarker spots down-regulated in the resistant patient bone marrow aspirate (2319; 2414; 2417; 2418; and 2421) are indicated.

FIG. 2: Graphical and numerical comparison of the average concentrations of the 5 biomarker protein spots identified as beta- and/or gamma-actin in the bone marrow aspirate samples from 6 imatinib mesylate resistant and 9 sensitive CML patients. Also shown are the averages for each biomarker and the ratio/fold increase of the averages of the sensitive to the resistant patient groups.

FIG. 3: The proposed role for the loss of beta- and/or gamma-Actin in imatinib mesylate resistant chronic myelogenous leukemia, showing the interaction of gamma- and/or beta-Actin with BCR-Ab1, the imatinib mesylate drug target and evidence for involvement of this interaction in facilitating imatinib mesylate binding to BCR-Ab1.

Table 1: Biochemical characteristics of the proteins in the 2D gel electrophoresis standard mixture.

Table 2: The major tryptic peptides identified by MaldiTOF MS as belonging to beta- and gamma-actin, including a peptide specific for gamma- and beta-Actin, not found in alpha-Actin.

Table 3: Computer readable form of amino acid sequence listing of gamma-(Sequence 1) and beta-actin (Sequence 2).

### DESCRIPTION OF THE PREFERRED EMBODIMENTS

The present invention is a diagnostic assay for differentiating cancer patients having the capacity to respond to treatment with an Ab1 kinase inhibitor from patients potentially resistant to an Ab1 kinase inhibitor, and a drug target and method for rational design of a drug design for overcoming resistance to treatment with an Ab1 kinase inhibitor. The method is based on the use of two-dimensional (2D) gel electrophoresis to separate the complex mixture of proteins found in bone marrow aspirates from patients with chronic myelogenous leukemia, and the quantitation of a group of identified biomarkers to differentiate between chronic myelogenous leukemia patients having the capacity to respond to treatment with the Ab1 kinase inhibitor, imatinib mesylate, and chronic myelogenous leukemia patients potentially resistant to treatment with the Ab1 kinase inhibitor, imatinib mesylate.

In the context of the present invention CML consists of bone marrow aspirate biopsy diagnosed CML.

In the context of the present invention, the “protein expression profile” corresponds to the steady state level of the various proteins in biological samples that can be expressed quantitatively. These steady state levels are the result of the combination of all the factors that control protein concentration in a biological sample. These factors include but are not limited to: the rates of transcription of the genes encoding the hnRNAs; processing of the hnRNAs into mRNAs; The rates of splicing and the splicing variations during the processing of the hnRNAs into mRNAs which govern the relative amounts of the protein sequence isoforms; the rates of processing of the various mRNAs by 3′-polyadenylation and 5′-capping; the rates of transport of the mRNAs to the sites of protein synthesis; the rates of translation of the mRNA's into the corresponding proteins; the rates of protein post-translational modifications, including but not limited to phosphorylation, nitrosylation, methylation, acetylation, glycosylation, poly-ADP-ribosylation, ubiquitinylation, and conjugation with ubiquitin-like proteins; the rates of protein turnover via the ubiquitin-proteosome system and via proteolytic processing of the parent protein into various active and inactive subcomponents; the rates of intracellular transport of the proteins among compartments, such as but not limited to the nucleus, the cytoplasm, lysosomes, golgi, the cell membrane, the endoplasmic reticulum, and the mitochondrion; the rates of secretion of the proteins into the interstitial space; the rates of secretion related protein processing; and the stability and rates of proteolytic processing and degradation of the proteins in the biological sample before and after the sample is taken from the patient.

In the context of the present invention, a “biomarker” corresponds to a protein present in a biological sample from a patient, wherein the quantity of the biomarker in the biological sample provides information about whether the patient exhibits an altered biological state such as the potential to respond to or resist a particular drug treatment.

The method of the present invention is based on the quantification of specified proteins. Preferably the proteins are separated and identified by 2D gel electrophoresis. In the past, this method has been considered highly specialized, labor intensive and non-reproducible.

Only recently with the advent of integrated supplies, robotics, and software combined with bioinformatics has progression of this proteomics technique in the direction of diagnostics become feasible. The promise and utility of 2D gel electrophoresis is based on its ability to detect changes in protein expression and to discriminate protein isoforms that arise due to variations in amino acid sequence and/or post-synthetic protein modifications such as proteolytic processing, phosphorylation, nitrosylation, ubiquitination, conjugation with ubiquitin-like proteins, acetylation, and glycosylation. These are important variables in cell regulatory processes involved in disease states.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between serial samples from the same individual or between samples from several individuals.

### Materials and Methods:

### Sample Collection and Preparation

Bone marrow aspirate samples were acquired by needle aspiration, centrifuged at 1200×g for 15 minutes, and the cells were frozen at −80° C. or below until shipment. Samples were shipped on dry ice and were delivered within 24 hours of shipping.

Once the samples were received, logged in, and assigned a sample number; they were further processed in preparation for 2D gel electrophoresis. All samples were stored at −80° C. or below. When the samples were removed from storage, they were placed on ice for thawing and kept on ice for further processing.

### Separation of Proteins in Patient Samples

The bone marrow aspirate proteins protein from chronic myelogenous leukemia patients analyzed in the present invention were separated using 2D gel electrophoresis. Other various techniques known in the art for separating proteins can also be used. These other techniques include but are not limited to gel filtration chromatography, ion exchange chromatography, reverse phase chromatography, affinity chromatography, or any of the various electrophoresis and centrifugation techniques well known in the art. In some cases, a combination of one or more chromatography, electrophoresis or centrifugation steps may be combined via electrospray or nanospray with mass spectroscopy or tandem mass spectroscopy, or any protein separation technique that determines the pattern of proteins in a mixture either as a one-dimensional, two-dimensional, three-dimensional or multi-dimensional pattern or list of proteins present.

### Two Dimensional Gel Electrophoresis of Samples

Preferably the protein profiles of the present invention are obtained by subjecting biological samples to two-dimensional (2D) gel electrophoresis to separate the proteins in the biological sample into a two-dimensional array of protein spots.

Two-dimensional gel electrophoresis is a useful technique for separating complex mixtures of proteins and can be performed using a variety of methods known in the art (see, e.g., U.S. Pat. Nos. 5,534,121; 6,398,933; and 6,855,554).

Preferably, the first dimensional gel is an isoelectric focusing gel and the second dimension gel is a denaturing polyacrylamide gradient gel.

Proteins are amphoteric, containing both positive and negative charges and like all ampholytes exhibit the property that their charge depends on pH. At low pH (acidic conditions), proteins are positively charged while at high pH (basic conditions) they are negatively charged. For every protein there is a pH at which the protein is uncharged, the protein's isoelectric point. When a charged molecule is placed in an electric field it will migrate towards the opposite charge.

In a pH gradient such as those used in the present invention, containing a reducing agent such as dithiothreitol (DTT), a protein will migrate to the point at which it reaches its isoelectric point and becomes uncharged. The uncharged protein will not migrate further and stops. Each protein will stop at its isoelectric point and the proteins can thus be separated according to their isoelectric points. In order to achieve optimal separation of proteins, various pH gradients may be used. For example, a very broad range of pH, from about 3 to 11 or 3 to 10 can be used, or a more narrow range, such as from pH 4 to 7 or 5 to 8 or 7 to 10 or 6 to 11 can be used. The choice of pH range is determined empirically and such determinations are within the skill of the ordinary practitioner and can be accomplished without undue experimentation.

In the second dimension, proteins are separated according to molecular weight by measuring mobility through a uniform or gradient polyacrylamide gel in the detergent sodium dodecyl sulfate (SDS). In the presence of SDS and a reducing agent such as dithiothreitol (DTT), the proteins act as though they are of uniform shape with the same charge to mass ratio. When the proteins are placed in an electric field, they migrate into and through the gel from one edge to the other. As the proteins migrate though the gel, individual proteins move at different speeds with the smaller ones moving faster than the larger ones, due to the sieving effect of the polyacrylamide gel. This process is stopped when the fastest moving components reach the other side of the gel. At this point, the proteins are distributed across the gel with the higher molecular weight proteins near the origin and the low molecular weight proteins near the other side of the gel.

It is well known in the art that various concentration gradients of acrylamide may be used for such protein separations. For example, a gradient of from about 5% to 20% may be used in certain embodiments or any other gradient that achieves a satisfactory separation of proteins in the sample may be used. Other gradients would include but not be limited to from about 5 to 18%, 6 to 20%, 8 to 20%, 10 to 20%, 8 to 18%, 8 to 16%, 10 to 16%, or any range as determined by one of skill.

The end result of the 2D gel procedure is the separation of a complex mixture of proteins into a two dimensional array, a pattern of protein spots, based on the differences in their individual characteristics of isoelectric point and molecular weight.

### Reagents

Protease inhibitor cocktail was from Roche Diagnostics Corporation (Indianapolis, Ind.), Protein assay and purification reagents were from Bio-Rad Laboratories (Hercules, Calif.). Immobilon-P membranes and ECL reagents were from Pierce (Rockford, Ill.). All other chemicals were from Sigma Chemical (St. Louis, Mo.).

### 2D Gel Standards

Purified proteins having known characteristics are used as internal and external standards and as a calibrator for 2D gel electrophoresis. The standards consist of seven reduced, denatured proteins that can be run either as spiked internal standards or as external standards to test the suitability of the gel electrophoresis run and reproducibility of the gels. A set mixture of proteins (the “standard mixture”) is used to determine pH gradients and molecular weights for the two dimensions of the electrophoresis operation. Table 1 lists the isoelectric point (pI) values and molecular weights for the proteins included in a standard mixture.

TABLE 1

| Protein | pI | Molecular Weight (Da) |
|---|---|---|
| Hen egg white conalbumin | 6.0, 6.3, 6.6 | 76,000 |
| Bovine serum albumin | 5.4, 5.5, 5.6 | 66,200 |
| Bovine muscle actin | 5.0, 5.1 | 43,000 |
| Rabbit muscle GAPDH | 8.3, 8.5 | 36,000 |
| Bovine carbonic anhydrase | 5.9, 6.0 | 31,000 |
| Soybean trypsin inhibitor | 4.5 | 21,500 |
| Equine myoglobin conalbumin | 7.0 | 17,500 |

In addition, standard mixtures such as Precision Plus Protein Standards (Bio-Rad Laboratories), a mixture of 10 recombinant proteins ranging from 10-250 kD, are typically added as external molecular weight standards for the second dimension, or the SDS-PAGE portion of the system. The Precision Plus Protein Standards have an r² value of the R_{f} vs. log molecular weight plot of >0.99.

### Separation of Proteins in Serum Samples

An appropriate amount of isoelectric focusing (IEF) loading buffer was added to the bone marrow aspirate sample, incubated at room temperature and vortexed periodically until the cell pellet was dissolved to visual clarity. The samples were centrifuged briefly before a protein assay was performed on the sample.

Approximately 100 μg of the proteins were suspended in a total volume of 184 μL of IEF loading buffer containing 5 M urea, 2 M Thiourea, 1% CHAPS, 2% ASB-14, 0.25% Tween 20, 100 mM DTT, 1% ampholytes pH 3-10, 5% glycerol, 1×EDTA-free protease inhibitor cocktail and 1 μL Bromophenol Blue as a color marker to monitor the process of gel electrophoresis. Each sample was loaded onto an 11 cm IEF strip (Bio-Rad Laboratories), pH 4-7, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad Laboratories running buffer lx Tris-Glycine-SDS.

The washed IEF strips were then laid on the surface of Bio Rad pre-cast CRITERION SDS-gels 10-20%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

### Staining and Analysis of the 2D Gels

Once the 2D gel patterns of the patient samples were obtained, the protein spots resolved in the gels were visualized with either a fluorescent or colored stain. In the preferred embodiment, the fluorescent dye SyproRuby™ (Bio-Rad Laboratories) was the stain. Once the protein spots had been stained, the gels were scanned by a digital fluorescent scanner, or when visible dyes such as Coomassie blue are employed a digital visible light scanner, and a digital image of the protein spot patterns of the gels were obtained, i.e. the protein expression profiles of the samples.

The digital image of the scanned gel was processed using PDQuest™ (Bio-Rad Laboratories) image analysis software to first detect the proteins, locate the selected biomarkers, and then to quantitate the protein in each of the selected spots. The scanned image was cropped and filtered to eliminate artifacts using the image editing control. Individual cropped and filtered images were then placed in a matched set for comparison to other images and controls.

This process allowed quantitative and qualitative spot comparisons across gels and the determination of protein biomarker molecular weight and isoelectric point values. Multiple gel images were normalized to allow an accurate and reproducible comparison of spot quantities across two or more gels. The gels were normalized using the “total of all valid (detected and confirmed by the operator) spots method” in that a small percentage of the 1200 protein spots detected and verified change between samples, and that all spots detected and verified is a good estimate to correct for any differences in total protein amount applied to each gel. The quantitative amounts of the selected biomarkers present in each sample were then exported for further analysis using mathematical, graphical, and statistical programs.

### Tryptic Digestion, MALDI/MS, and LC-MS/MS

Following software analysis, unique spots were excised from the gel using the ProteomeWorks™ robotic spot cutter (Bio-Rad). In-gel spots were subjected to proteolytic digestion on a ProGest™ (Genomic Solutions, Ann Arbor, Mich.). A portion of the resulting digest supernatant was used for MaldiTOF MS analysis. Peptide solutions were concentrated and desalted using μ-C18 ZipTips™ (Millipore). Peptides were eluted with MaldiTOF MS matrix alpha-cyano 4-hydroxycinnamic acid prepared in 60% acetonitrile, 0.2% TFA. Samples were robotically spotted onto the MaldiTOF MS chip, using ProMS™ (Genomic Solutions, Ann Arbor, Mich.).

MaldiTOF MS data was acquired on an Applied Biosystems Voyger DE-STR instrument and the observed m/z values were submitted to ProFound (Proteometrics software package) for peptide mass fingerprint searching using NCBInr database. The spectrum of all masses submitted to the database were first verified for appropriate signal to noise and protein identities were based upon the best fit containing the most abundant peptides.

### Analysis of Samples

### Characterization of a Subject as Sensitive or Resistant to Imatinib Treatment

A chronic myelogenous leukemia patient is regarded as having responded to treatment if within 12 months of starting treatment, no Philadelphia-chromosome positive cells are observed on examination of 30 bone marrow metaphases.

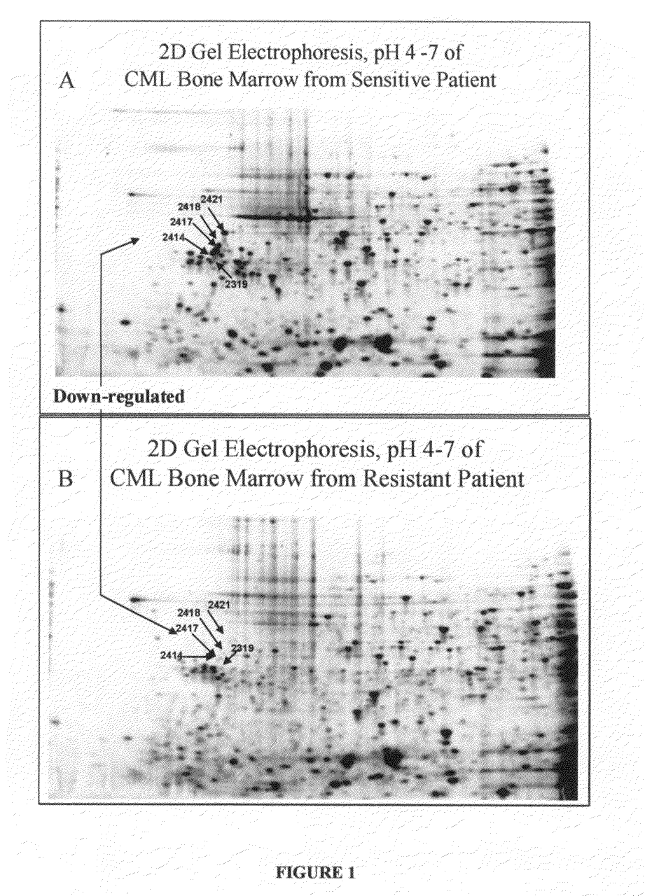

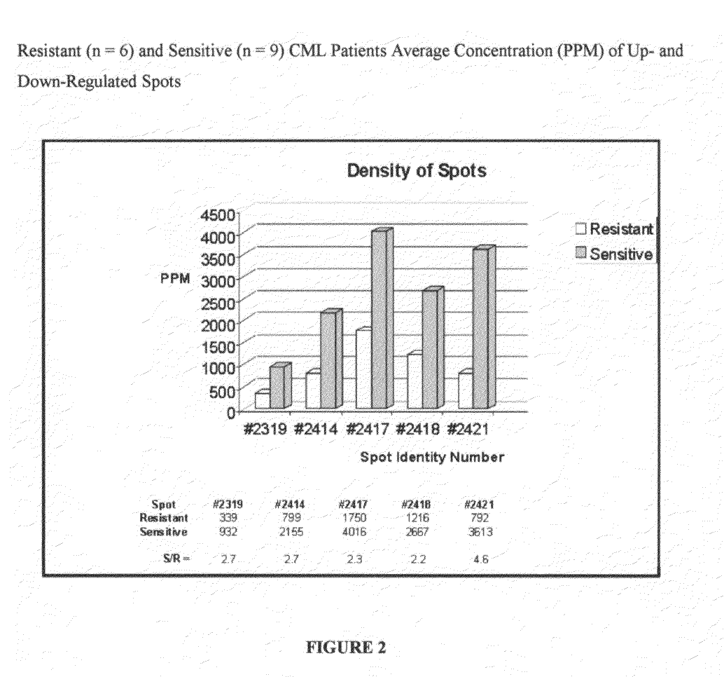

Representative samples from individuals with known cases of chronic myelogenous leukemia, some of whom subsequently responded to treatment with the Ab1 kinase inhibitor, imatinib mesylate, and some of whom did not respond, were subjected to 2D gel electrophoresis and the digital images compared to find differences in patterns of expression of proteins predictive of sensitivity or resistance to the Ab1 kinase inhibitor, Imatinib mesylate. The spot locations for the selected 5 protein biomarker spots consistently down regulated in bone marrow aspirates from patients subsequently found to be resistant to imatinib mesylate are illustrated in FIG. 1 and the average differences in concentration of the biomarkers are illustrated in FIG. 2.

The beta- and gamma-Actin isoforms represented by these five spots (#2319, #2414, #2417, #2418, #2421, FIGS. 1 and 2), were down-regulated in imatinib mesylate resistant patients in that the cluster of 5 protein spots demonstrated a 2.3-4.6 fold elevation on average in the bone marrows of the patients sensitive to Imatinib when compared to the samples from the Imatinib resistant patients. These proteins were identified as beta- and gamma-Actins (Tables 2 and 3). The identification of these 5 spots as the cytoskeleton microfilament beta- and gamma-Actins is based upon Maldi-TOF MS of in-gel digests, wherein only the most abundant peptides (>3000 cts), with clearly discernable spectra above background, were submitted for database searching and where the identifications also contained the most abundant peptide hits. This approach identified only beta- and gamma-Actin peptides for spots (#2319, #2414, #2417, #2418, #2421, Tables 2 and 3). Amino terminal Edman degradation of the proteins after electrophoretic transfer to membranes indicated the proteins were blocked, which is known to be the case for gamma- and beta-Actins.

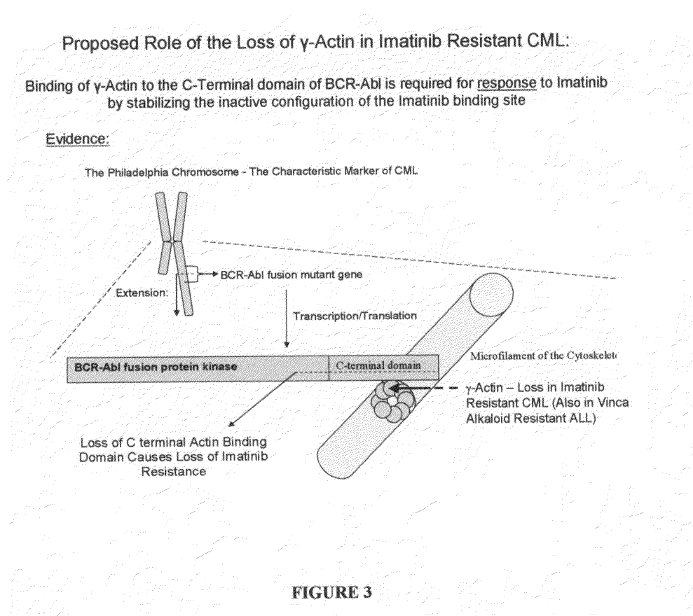

The gamma- and beta-Actins interact directly with the imatinib mesylate drug target, BCR-Ab1, which is know to bind to them via its COOH-terminal domain (Underhill-Day N, et al. 2006, British Journal of Hematology 132: 774-783.). Interestingly, a pronounced down-regulation of gamma-Actin also accompanies Vincristine resistance in acute lymphocytic leukemia (ALL) (Virrills N M, et al. 2006, Proteomics 6: 1681-1694). The implications of these results in the light of the literature are shown in FIG. 3, indicating that the actin binding site on BCR-Ab1 is a likely target for a drug mimicking the action of beta- and/or gamma Actin at their binding site on BCR-Ab1.

The bone marrow aspirate samples may also be subjected to various other techniques known in the art for separating and quantitating proteins. Such techniques include, but are not limited to gel filtration chromatography, ion exchange chromatography, reverse phase chromatography, affinity chromatography (typically in an HPLC or FPLC apparatus), or any of the various electrophoresis or centrifugation techniques well known in the art. Certain embodiments would also include a combination of one or more chromatography; capillary electrophoresis or centrifugation steps combined via electrospray or nanospray with mass spectrometry or tandem mass spectrometry of the proteins themselves, or of a total digest of the protein mixtures. Certain embodiments may also include surface enhanced laser desorption mass spectrometry or tandem mass spectrometry, or any protein separation technique that determines the pattern of proteins in the mixture either as a one-dimensional, two-dimensional, three-dimensional or multi-dimensional protein pattern, and or the pattern of protein amino acid sequence isoforms or post synthetic modification isoforms.

Quantitation of a protein by antibodies directed against that protein is well known in the field. The techniques and methodologies for the production of one or more antibodies to the proteins, routine in the field and are not described in detail herein.

As used herein, the term antibody is intended to refer broadly to any immunologic binding agent such as IgG, 1 gM, IgA, IgD and IgE. Generally, IgG and/or 1 gM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of human, murine, monkey, rat, hamster, rabbit and even chicken origin. Due to the ease of preparation and ready availability of reagents, murine monoclonal antibodies are generally preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has 20 an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the one or more of the 5 protein biomarkers may be used in a variety of assays in order to quantitate the protein in bone marrow aspirate samples, or other fluid or tissue samples. Well known methods include immunoprecipitation, antibody sandwich assays, ELISA and affinity chromatography methods that include antibodies bound to a solid support. Such methods also include micro arrays of antibodies or proteins contained on a glass slide or a silicon chip, for example.

It is contemplated that arrays of antibodies containing to up to 5 protein biomarkers, or peptides derived, may be produced in an array and contacted with the bone marrow aspirate samples or protein fractions thereof in order to quantitate the proteins. The use of such micro arrays is well known in the art and is described, for example in U.S. Pat. No. 5,143,854, incorporated herein by reference.

The present invention includes a screening assay for the potential of cancer patients to respond or to resist treatment with an Ab1 kinase inhibitor, based on the concentration of the 5 gamma- and/or beta-Actin protein biomarkers. One embodiment of the assay will be constructed with antibodies recognizing up to 5 protein gamma- and/or beta-Actin biomarkers. One or more antibodies targeted to antigenic determinants of up to 5 gamma- and/or beta-Actin protein biomarkers will be spotted onto a surface, such as a polyvinyl membrane or glass slide. As the antibodies used will each recognize an antigenic determinant of up to 5 protein gamma- and/or beta-Actin biomarkers, incubation of the spots with patient samples will permit attachment of up to 5 gamma- and/or beta-Actin protein biomarkers to the antibody.

The binding of up to 5 gamma- and/or beta-Actin protein biomarkers can be reported using any of the known reporter techniques including radioimunoassays (RIA), stains, enzyme linked immunosorbant assays (ELISA), sandwich ELISAs with a horseradish peroxidase (HRP)-conjugated second antibody also recognizing up to 5 gamma- and/or beta-Actin protein biomarkers, the pre-binding of fluorescent dyes to the proteins in the sample, or biotinylating the proteins in the sample and using an HRP-bound streptavidin reporter. The HRP can be developed with a chemiluminescent, fluorescent, or colorimetric reporter. Other enzymes, such as luciferase or glucose oxidase, or any enzyme that can be used to develop light or color can be utilized at this step.

As shown in Table 2, one of the tryptic peptides found in the in-gel digests of the 5 gamma- and/or beta-Actin protein biomarkers is found in beta- and gamma-actin and not found in alpha-Actin. For high throughput immunoassays, biomarker specific antibodies can be developed using only the epitopes specific for the beta- and/or gamma-Actin. For example, peptides obtained by purification from tryptic digests, or made by solid phase peptide synthesis, containing that specific amino acid sequence can be used to immunize rabbits, sheep, chickens, or goats, for polyclonal antibodies, or mice to produce monoclonal antibodies either with classic hybridoma technologies or phage display methods.

Alternatively, peptides containing the amino acid sequence of the portion of gamma and/or beta actin that binds to an Ab1 kinase can be used to mimic the Ab1 kinase binding action of gamma and/or beta-Actin and therefore render resistant cancer sensitive to an Ab1 kinase inhibitor, when used in combination with an Ab1 kinase inhibitor, to enable the Ab1 kinase inhibitor to kill the resistant cancer cells. A drug with such an Ab1 kinase activity enhancing capacity may also help to reduce the potential for recurrence of the cancer.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention.

It is also well recognized in the art that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A protein or group of up to 5 proteins associated with sensitivity or resistance to an Ab1 kinase inhibitor.

2. The protein or group of proteins of claim 1, wherein the protein or group of proteins are gamma-Actins.

3. The protein or group of proteins of claim 1, wherein the protein or group of proteins are beta-Actins.

4. The group of proteins of claim 1, wherein the group of proteins are gamma- and beta-Actins.

5. The protein or group of proteins of claim 1, wherein the Ab1 kinase inhibitor is imatinib mesylate

6. The protein or group of proteins of claim 1, wherein a reduced quantity of up to five of the proteins is associated with resistance to an Ab1 kinase inhibitor.

7. The protein or group of proteins of claim 1, wherein the binding of the protein to an Ab1 kinase is associated with sensitivity of an Ab1 kinase to an Ab1 kinase inhibitor.

8. The protein of claim 7, wherein a reduced quantity of the protein is associated with an increase in Ab1 kinase not bound to the protein, wherein the Ab1 kinase not bound to the protein is resistant to the Ab1 kinase inhibitor.

9. The protein or group of proteins of claim 1 wherein the Ab1 kinase inhibitor is being used to treat cancer.

10. The protein of claim 9 wherein the cancer is a Philadelphia chromosome positive cancer.

11. The protein of claim 9, wherein the cancer is a hematological malignancy such as leukemia, Hodgkin's lymphoma, Non-Hodgkin's lymphoma, myeloma, or meylodisplastic syndrome.

12. The protein of claim 11 wherein the leukemia is acute myelogenous leukemia, chronic myelogenous leukemia, acute lymphocytic leukemia, or chronic lymphocytic leukemia

13. The protein of claim 9 wherein the cancer is lung cancer.

14. The protein of claim 9 wherein the cancer is gastrointestinal stromal cancer.

15. A method for diagnosing the potential for resistance to an Ab1 kinase inhibitor comprising:
a. Obtaining a biological sample from a patient
b. Measurement of the concentration of up to 5 gamma- and/or beta-Actin proteins in the biological sample,
c. Wherein a reduced concentration of up to 5 gamma- and/or beta-Actin proteins (compared to their concentrations from potentially sensitive or responding patients to Ab1 kinase inhibitor, FIG. 2) indicates a potential for the patient to resist an Ab1 kinase inhibitor.

16. A drug design for enhancing the response to an Ab1 kinase inhibitor, comprising:
a. Preparing a peptide containing a portion of the amino acid sequence of gamma- and/or beta-Actin that binds to the actin binding site of an Ab1 kinase,
b. Employing the peptide as a drug that is used in conjunction with an Ab1 kinase inhibitor,
c. Treatment of a patient predicted by the method of claim 15 to be resistant to the Ab1 kinase, whereby
d. The peptide binds to the Ab1 kinase and enhances the binding of the Ab1 kinase inhibitor, such that
e. The resistance due to a decreased quantity of gamma- and/or beta-Actin is overcome by the peptide mimicking the action of gamma- and/or beta-Actin in binding to the Ab1 kinase, thereby
f. Enhancing the binding of the Ab1 kinase inhibitor to the Ab1 kinase, thereby
g. Rendering the Ab1 kinase inhibitor resistant cancer sensitive to treatment with an Ab1 kinase inhibitor.

17. The method of claim 15, wherein the concentration of up to 5 gamma- and/or beta-Actin proteins is determined by 2D gel electrophoresis.

18. The method of claim 15, wherein the concentration of up to 5 gamma and/or beta actin proteins is determined by an immunoassay using antibodies specific to gamma- and/or beta-Actin.

19. The method of claim 16 wherein the peptide is one or more of the tryptic peptides in Table 2.

20. The peptide or peptides of claim 19 wherein the peptide or peptides is/are prepared by trypsin digestion of the gamma- and/or beta-Actin, and is/are purified from the trypsin digest.

21. The method of claim 16 wherein the peptide is one or more of peptides derived from the amino acid sequences in Table 3.

22. The peptide of claim 16, wherein the peptide is purified by affinity chromatography using an immobilized portion of the actin binding domain of an Ab1 kinase.

23. The method of claim 16, wherein the peptide or peptides are prepared by solid phase peptide synthesis.

24. The method of claim 16, wherein the peptide is prepared by expression in a recombinant system.


SEQUENCE LISTING (2 sequences)

SEQ ID NO: 1 - protein, 375 aa, Homo sapiens
  INIT_MET (1)..(1): Amino terminal amino acid Met (1) removed to make mature protein beginning with acetyl-NH-Glu (2) blocked amino terminus
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 2 - protein, 375 aa, Homo sapiens
  INIT_MET (1)..(1): Amino terminal amino acid Met (1) removed to make mature protein beginning with acetyl-NH-Glu (2) blocked amino terminus
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF